(19) United States
(12) Patent Application Publication
(10) Pub. No.: US 2026/0283935 A1
(43) Pub. Date: Sep. 24, 2026

(54) Cosmetic Compositions Comprising A UV Filter And A PEG-Containing Dimethicone Crosspolymer

(71) Applicant: ELC Management LLC, Melville, NY (US)

(72) Inventors: Wei Chen, Syosset, NY (US); Snehal M. Shah, Nesconset, NY (US); Elena Sava, Hewlett, NY (US); Victoria Brady, West Babylon, NY (US); Geng Li, Brooklyn, NY (US); James Lyons, Holtsville, NY (US); Monica Apostol, South Setauket, NY (US)

(21) Appl. No.: 19/083,025

(22) Filed: Mar. 18, 2025

(51) Int. Cl. A61K 8/894; A61Q 17/04

(52) U.S. Cl. CPC A61K 8/894; A61Q 17/04; A61K 2800/31; A61K 2800/592; A61K 2800/594

(57) ABSTRACT

Cosmetic compositions including a cosmetically acceptable base, at least one UV filter, a polyethylene glycol (PEG)-containing dimethicone crosspolymer, and an additional elastomer are disclosed. The cosmetic compositions are preferably anhydrous. The at least one UV filter preferably includes at least one organic UV filter. The combination of the at least one organic UV filter and the PEG-containing dimethicone crosspolymer exhibit an unexpected synergistic relationship advantageously improving a photoprotective efficacy (e.g., sun protection factor (SPF), UVA protection factor (UVAPF)), of the cosmetic compositions including the organic UV filter and the PEG-containing dimethicone crosspolymer.

### FIELD OF THE INVENTION

The disclosure, in various embodiments, relates generally to the field of photoprotective cosmetic or dermatological compositions. More particularly, the disclosure relates to cosmetic compositions comprising at least one UV filter, a polyethylene glycol (PEG)-containing dimethicone crosspolymer, and an additional elastomer.

### BACKGROUND

Exposure to ultraviolet (UV) radiation from the sun is a leading cause of skin damage, premature against, and an increased risk of skin cancer. Sunscreen compositions have been developed to protect the skin by absorbing, reflecting, and/or scattering UV radiation. These compositions typically contain organic UV filters, inorganic UV filters (e.g., particulates), or a combination thereof to achieve broad-spectrum protection.

While existing sunscreens and sunscreen compositions offer varying degrees of photoprotection, challenges remain in enhancing their efficacy, stability, and overall performance. Many compositions require high concentrations of UV filters to achieve sufficient sun protection factor (SPF) levels, which can lead to issues such as poor skin feel, increased skin irritation potential, and degradation of aesthetic properties, resulting in an ashen or pasty appearance. Consumers desire sunscreens that provide effective protection while maintaining desirable cosmetic properties.

Based on the foregoing, there is a need for a photoprotective cosmetic composition that enhances photoprotection efficacy while addressing stability, sensory attributes, and consumer preferences. In particular, there is a need for a composition which provides a combination of ingredients that synergistically boost the effectiveness of UV protection, improve stability, and result in a desirable sensory feel, providing an optimized balance of protection and aesthetic appeal.

### BRIEF SUMMARY

Cosmetic compositions including a cosmetically acceptable base, at least one organic UV filter, a polyethylene glycol (PEG)-containing dimethicone crosspolymer, and an additional elastomer are disclosed herein. The cosmetic compositions may be anhydrous. The additional elastomer may be substantially different from the PEG-containing dimethicone crosspolymer. The compositions may include the PEG-containing dimethicone crosspolymer in an amount within a range of from about 1% by weight (wt %) to about 25 wt % based on a total weight of the composition.

Photoprotective cosmetic compositions including a cosmetically acceptable base, at least one organic UV filter, a PEG-containing dimethicone crosspolymer, and a dimethicone crosspolymer substantially free of PEG are disclosed herein. The compositions may include the PEG-containing dimethicone crosspolymer in an amount within a range of from about 2 wt % to about 16 wt % based on a total weight of the composition.

Anhydrous cosmetic compositions including a cosmetically acceptable base, at least one organic UV filter, dimethicone/PEG-10/15 crosspolymer, and dimethicone/vinyl dimethicone crosspolymer are disclosed herein. The composition may exhibit an SPF of at least about 15.

### BRIEF DESCRIPTION OF THE DRAWINGS

FIG. 1 illustrates absorption spectra for a range of wavelengths between 290 nanometers (nm) and 400 nm for exemplary samples S1 and S2 and comparative samples C1 and C2 described in Example 3;

FIG. 2 illustrates absorption spectra for a range of wavelengths between 290 nm and 400 nm for exemplary sample S3 and comparative sample C3 described in Example 4; and

FIG. 3 illustrates absorption spectra for a range of wavelengths between 290 nm and 400 nm for exemplary samples S4-S6 and comparative sample C4 described in Example 5.

### DETAILED DESCRIPTION

The following description provides specific details, such as material types and process conditions, in order to provide a thorough description of embodiments described herein. However, a person of ordinary skill in the art will understand that the embodiments disclosed herein may be practiced without employing these specific details. Indeed, the embodiments disclosed herein may be practiced in conjunction with conventional formulation techniques employed in the cosmetic formulation industry. In addition, the description provided herein does not form a complete description of a cosmetic composition or a complete process flow for manufacturing a cosmetic composition. Only those elements and ingredients necessary to understand the embodiments described herein are described in detail below. Additional cosmetically acceptable ingredients may be included to form a complete cosmetic composition, without departing from the scope of this disclosure.

As used herein, the singular forms “a,” “an,” and “the” are intended to include the plural forms as well, unless the context clearly indicates otherwise.

As used herein, “and/or” includes any and all combinations of one or more of the associated listed items.

As used herein, “at least one” means and includes one or more (e.g., two, three, multiple, any amount greater than or equal to one).

As used herein, “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 90.0% to 110.0% of the numerical value, such as within a range of from 95.0% to 105.0% of the numerical value, within a range of from 97.5% to 102.5% of the numerical value, within a range of from 99.0% to 101.0% of the numerical value, within a range of from 99.5% to 100.5% of the numerical value, or within a range of from 99.9% to 100.1% of the numerical value.

The dimensions, amounts, and values disclosed herein are not to be understood as being strictly limited to the exact numerical values recited. Instead, unless otherwise specified, each such dimension, amount, and value is intended to mean both the recited value and a functionally equivalent range surrounding that value. For example, an amount disclosed as “35% by weight” is intended to mean “about 35% by weight.”

As used herein, the term “substantially” in reference to a given parameter, property, or condition means and includes to a degree that one of ordinary skill in the art would understand that the given parameter, property, or condition is met with a degree of variance, such as acceptable tolerances. By way of example, depending on the particular parameter, property, or condition that is substantially met, the parameter, property, or condition may be at least 90.0% met, at least 95.0% met, at least 99.0% met, at least 99.9% met, or even 100.0% met.

As used herein, the term “anhydrous” in reference to a material or composition means and includes that the anhydrous material or composition comprises less than or equal to 10% by weight (wt %), less than or equal to 5 wt %, less than or equal to 1 wt %, or less than or equal to 0.01 wt % of water based on a total weight of the material or composition or even equal to 0 wt % of water based on a total weight of the material or composition.

As used herein, the term “UV filter” means and includes a compound or an active that has photoprotection efficacy. The UV filter may be an organic (e.g., chemical, carbon-based) UV filter or an inorganic (e.g., physical, carbon-free) UV filter. As used herein, the term “UVA filter” means and includes a compound or an active that blocks at least a portion of UV radiation in the wavelength range of from about 320 nm to about 400 nm. As used herein, the term “UVB filter” means and includes a compound or an active that blocks at least a portion of UV radiation in the wavelength range of from about 290 nm to about 320 nm. As used herein, the term “UVA/B filter” means and includes a compound or an active that blocks at least a portion of UV radiation in the wavelength range of from about 320 nm to about 400 nm and at least a portion of UV radiation in the wavelength range of from about 290 nm to about 320 nm.

As used herein, the term “sunscreen” means and includes a composition comprising at least one UV filter.

As used herein, the term “silicone-alternative elastomer” means and includes natural and/or synthetic elastomers that are free of silicones (e.g., silicone polymers).

As used herein, the term “cosmetically acceptable” means that the given composition, formulation, or component is generally suitable for use in contact with human keratinous tissue (e.g., skin, hair) without undue toxicity, incompatibility, instability, allergic response, hazardous reaction, and the like.

Cosmetic compositions according to embodiments of the disclosure include a cosmetically acceptable base, at least one UV filter, a polyethylene glycol (PEG)-containing dimethicone crosspolymer, and an additional elastomer. The cosmetic compositions are preferably anhydrous. The at least one UV filter preferably includes at least one organic UV filter. The combination of the at least one organic UV filter and the PEG-containing dimethicone crosspolymer exhibit an unexpected synergistic relationship advantageously improving a photoprotective efficacy (e.g., sun protection factor (SPF), UVA protection factor (UVAPF)), of the cosmetic compositions including the organic UV filter and the PEG-containing dimethicone crosspolymer. In some embodiments, the cosmetic compositions may be formulated to exhibit an SPF value of at least about 30. The additional elastomer may advantageously improve the stability of the cosmetic compositions (e.g., anhydrous cosmetic compositions), preventing phase separation while maintaining a desired texture, color, and effectiveness, thereby extending a shelf-life of the cosmetic compositions. The combination of the PEG-containing dimethicone crosspolymer and the additional elastomer may advantageously result in preferable sensory characteristics (e.g., tactile, visual appearance, texture, consistency, application, spreadability, olfactory, afterfeel, etc.) of the cosmetic compositions, improving performance of the cosmetic compositions and improving a consumer's experience during and/or after use (e.g., application) of the compositions.

The cosmetic compositions disclosed herein may be in the form of an emulsion (e.g., oil-in-water, water-in-oil, oil-in-oil, silicone-in-water, silicone-in-oil, water-in-silicone, oil-in-silicone, water-in-oil-in-water, oil-in-water, oil-in-water-in-oil, oil-in-water-in-silicone, oil-in-silicone-in-oil, etc.), a cream, a lotion, a solution, an anhydrous base (e.g., lipstick, powders), an anhydrous multi-phase system, a gel, an ointment, a paste, a liquid, an oil, a butter, a stick, a spray, an aerosol, a solid, or any other suitable form. The compositions disclosed herein are preferably anhydrous. The compositions disclosed herein more preferably include less than 5 wt % of water, based on a total weight of the composition. The compositions disclosed herein most preferably include about 0 wt % (e.g., are substantially free) or equal to 0 wt % (e.g., are free) of water, based on a total weight of the composition. The compositions disclosed herein are preferably in the form of an anhydrous system (e.g., anhydrous emulsion, anhydrous base, anhydrous multi-phase system, etc.).

The compositions disclosed herein include at least one UV filter. The at least one UV filter may include a UVA filter, a UVB filter, a UVA/B filter, or any combination thereof. The at least one UV filter may include one or more organic UV filters (e.g., chemical UV filters, UV absorbers), one or more inorganic UV filters (e.g., physical UV filters, UV blockers, UV reflectors), or any combination thereof. The at least one UV filter preferably includes at least one organic UV filter. In some embodiments, the compositions are substantially free of inorganic UV filters. The at least one UV filter is preferably dispersible within an anhydrous composition (e.g., system). Any and all UV filters typical in the art of sunscreen formulation are considered to be suitable.

By way of non-limiting example, the one or more organic UV filters may be selected from octocrylene, butyl methoxydibenzoylmethane (avobenzone), ethylhexyl salicylate (octisalate), homosalate, benzophenone-3 (oxybenzone), 4-methylbenzylidene camphor (enzacamene), benzophenone-4 (sulisobenzone), bis-ethylhexyloxyphenol methoxyphenyl triazine (bemotrizinol), diethylamino hydroxybenzoyl hexyl benzoate, diethylhexyl butamido triazone, disodium phenyl dibenzimidazole tetrasulfonate, drometrizole trisiloxane, ethylhexyl methoxycinnamate (octinoxate), ethylhexyl triazone, isoamyl p-methoxycinnamate (amiloxate), isopropyl methoxycinnamate, menthyl anthranilate (meradimate), methylene bis-benzotriazolyl tetramethylbutylphenol (bisoctrizole), para-aminobenzoic acid (PABA), ethylhexyl dimethyl PABA (padimate O), phenylbenzimidazole sulfonic acid (ensulizole), terephthalylidene dicamphor sulfonic acid, and combinations thereof. In some embodiments, the compositions include at least one organic UV filter. In some embodiments, the compositions include at least four organic UV filters. In some embodiments, the at least one UV filter includes one or more of octocrylene, avobenzone, octisalate, homosalate, and mixtures thereof. In some embodiments, the at least one UV filter consists of octocrylene, avobenzone, octisalate, and homosalate.

By way of non-limiting example, the one or more inorganic UV filters may include titanium dioxide, zinc oxide, or any combination thereof.

The compositions disclosed herein may include the one or more UV filters in a total (e.g., combined) amount within a range of from about 0.01 wt % to about 50 wt % based on a total weight of the composition, such as, for example, within a range of from about 0.1 wt % to about 25 wt %, from about 1 wt % to about 20 wt %, from about 1 wt % to about 25 wt %, from about 3 wt % to about 18 wt %, from about 5 wt % to about 15 wt %, from about 5 wt % to about 20 wt %, from about 10 wt % to about 30 wt %, or from about 10 wt % to about 50 wt % based on the total weight of the composition. Other amounts (e.g., total amounts) of the one or more UV filters that are typical in the art of sunscreen formulation or cosmetic formulation are also considered. In some embodiments, the compositions include octocrylene at an amount of about 9 wt %, avobenzone at an amount of about 3 wt %, octisalate at an amount of about 4.5 wt %, and homosalate at an amount of about 7 wt % based on the total weight of the composition.

The compositions disclosed herein include at least one polyethylene glycol (PEG)-containing dimethicone crosspolymer elastomer (e.g., PEG-modified dimethicone crosspolymer, PEG-containing dimethicone crosspolymer). By way of non-limiting example, the PEG-containing dimethicone crosspolymer may include PEG-12 dimethicone crosspolymer, PEG-10 dimethicone crosspolymer, dimethicone/PEG-10 crosspolymer, dimethicone/PEG-15 crosspolymer, dimethicone/PEG-10/15 crosspolymer, PEG-8, dimethicone/polysorbate 20 crosspolymer, PEG-10 dimethicone/vinyl dimethicone crosspolymer, PEG-10/lauryl dimethicone crosspolymer, PEG-15/lauryl dimethicone crosspolymer, PEG-15/lauryl polydimethylsiloxyethyl dimethicone crosspolymer, PEG-12 dimethicone/polypropylene glycol (PPG)-20 crosspolymer, PEG-12 dimethicone/bis-isobutyl PPG-20 crosspolymer, bis-vinyldimethicone/PEG-10 dimethicone crosspolymer, or any combination thereof. The at least one PEG-containing dimethicone crosspolymer preferably includes dimethicone/PEG-10/15 crosspolymer, PEG-12 dimethicone/PPG-20 crosspolymer, or a combination thereof. In some embodiments, the at least one PEG-containing dimethicone crosspolymer includes two or more PEG-containing dimethicone crosspolymers.

The compositions disclosed herein may include the at least one PEG-containing dimethicone crosspolymer in an amount ranging from about 1 wt % to about 25 wt % based on a total weight of the cosmetic composition, such as, for example, from about 1 wt % to about 20 wt %, from about 1 wt % to about 18 wt %, from about 1 wt % to about 15 wt %, from about 1 wt % to about 10 wt %, from about 1 wt % to about 5 wt %, from about 2 wt % to about 20 wt %, from about 2 wt % to about 16 wt %, or from about 5 wt % to about 15 wt % based on the total weight of the cosmetic composition. The compositions preferably include the at least one PEG-containing dimethicone crosspolymer in an amount ranging from about 2 wt % to about 16 wt % based on the total weight of the cosmetic composition, such as, for example, from about 6 wt % to about 15 wt %.

The at least one PEG-containing dimethicone crosspolymer may be in the form of a powder, a solution, a suspension, a dispersion, a paste, a gel, or any other suitable form. The at least one PEG-containing dimethicone crosspolymer may be provided within a cosmetically acceptable carrier. The PEG-containing dimethicone crosspolymer may be provided within the cosmetically acceptable carrier at an amount within a range of from about 10 wt % to about 60 wt % based on a total weight of the PEG-containing dimethicone crosspolymer and the cosmetically acceptable carrier, such as, for example, within a range of from about 10 wt % to about 30 wt %, from about 10 wt % to about 20 wt %, from about 15 wt % to about 35 wt %, from about 15 wt % to about 30 wt %, from about 15 wt % to about 40 wt %, from about 20 wt % to about 35 wt %, from about 20 wt % to about 40 wt %, from about 25 wt % to about 50%, or from about 30 wt % to about 60 wt % based on the total weight of the PEG-containing dimethicone crosspolymer and the cosmetically acceptable carrier. The compositions disclosed herein may include the PEG-containing dimethicone crosspolymer within the cosmetically acceptable carrier in a combined amount within a range of from about 10 wt % to about 60 wt % based on a total weight of the composition, such as, for example, from about 10 wt % to about 30 wt %, from about 10 wt % to about 40 wt %, from about 10 wt % to about 50 wt %, from about 15 wt % to about 30 wt %, from about 15 wt % to about 45 wt %, from about 20 wt % to about 30 wt %, from about 20 wt % to about 40 wt %, or from about 20 wt % to about 60 wt % based on the total weight of the composition. The compositions preferably include the PEG-containing dimethicone crosspolymer within the cosmetically acceptable carrier in a combined amount of from about 20 wt % to about 40 wt % based on the total weight of the composition.

Such PEG-containing dimethicone crosspolymers are commercially available from numerous sources including, but not limited to, Shin-Etsu Chemical Co., Ltd. (Tokyo, Japan), Dow Chemical Co. (Midland, MI), Access Ingredients, Inc. (South Pasadena, CA), Hunan Silok Silicone Co., Ltd. (Guangzhou, China), and ALZO International Inc. (Sayreville, NJ). The PEG-containing dimethicone crosspolymer may include, but is not limited to, KSG-210 (dimethicone (and) dimethicone/PEG-10/15 crosspolymer), KSG-270 (diphenylsiloxy phenyl trimethicone (and) dimethicone/PEG-10/15 crosspolymer), KSG-310 (mineral oil (and) PEG-15/lauryl dimethicone crosspolymer), KSG-320 (PEG-15/lauryl dimethicone crosspolymer (and) isododecane), KSG-330 (triethylhexanoin (and) PEG-15/lauryl dimethicone crosspolymer), or KSG-340 (squalane (and) PEG-10/lauryl dimethicone crosspolymer (and) PEG-15/lauryl dimethicone crosspolymer) from Shin-Etsu Chemical Co., Ltd, Dowsil™ EL-7040 (caprylyl methicone (and) PEG-12 dimethicone/PPG-20 crosspolymer) from Dow Chemical Co., AccessSIL EMUL-29 (dimethicone (and) dimethicone/PEG-10/15 crosspolymer) from Access Ingredients, Inc., Silicone Elastomer Gel SiCare® 9960 (dimethicone (and) PEG-10 dimethicone/vinyl dimethicone crosspolymer (and) polymethylsilsesquioxane) from Hunan Silok Silicone Co., Ltd., NuLastic™ Surfa ID-7 (isododecane (and) bis-vinyldimethicone/PEG-10 dimethicone crosspolymer), NuLastic™ Surfa D99-9, (isononyl isononanoate (and) bis-vinyldimethicone/PEG-10 dimethicone crosspolymer) or NuLastic™ Surfa DM-6 (dimethicone (and) bis-vinyldimethicone/PEG-10 dimethicone crosspolymer) from ALZO International Inc., or any combination thereof.

The compositions disclosed herein include at least one additional elastomer. The at least one additional elastomer is preferably different from the PEG-containing dimethicone crosspolymer. The at least one additional elastomer may include a silicone elastomer (e.g., polysiloxane, polysilicone), a non-silicone elastomer, such as, for example, polyisobutene, polyurethane, a natural rubber, or a block-copolymer rubber, or combinations thereof. The silicone elastomer may be crosslinked and formed by the reaction of vinyl terminated silicone (e.g., uni-, di-, or multi-vinyl terminated silicone) and hydrogen dimethicone. The vinyl terminated silicone may be bis-vinyldimethicone. By way of non-limiting example, the at least one additional elastomer may include polysilicone-11, a dimethicone crosspolymer, such as, dimethicone/vinyl dimethicone crosspolymer, dimethicone/PPG-20 crosspolymer, dimethicone/bis-isobutyl PPG-20 crosspolymer, or dimethicone polyglycerin-3 crosspolymer, a cross-linked polyurethane, such as, polyurethane-10, or polyurethane-100, a silicone-alternative elastomer, such as, for example, caprylic/capric triglyceride (and) dicaprylyl carbonate (and) silica (and) dextrin palmitate (and) diisostearyl malate (and) lactobacillus ferment, caprylic/capric triglyceride (and) dicaprylyl carbonate (and) dextrin palmitate (and) diisostearyl malate (and) lactobacillus ferment, or isoamyl laurate (and) decyl oleate (and) silica, or any combination thereof. Any and all cosmetically acceptable elastomers are considered to be suitable. In some embodiments, the at least one additional elastomer includes two or more additional elastomers. The at least one additional elastomer preferably includes a dimethicone crosspolymer. The at least one additional elastomer is preferably substantially free of PEG.

The compositions disclosed herein may include the at least one additional elastomer in an amount ranging from about 0.1 wt % to about 50 wt % based on a total weight of the cosmetic composition, such as, for example, from about 0.1 wt % to about 5 wt %, from about 0.1 wt % to about 8 wt %, from about 0.1 wt % to about 10 wt %, from about 1 wt % to about 5 wt %, from about 1 wt % to about 10 wt %, from about 1 wt % to about 15 wt %, from about 1 wt % to about 20 wt %, from about 1 wt % to about 30 wt %, or from about 1 wt % to about 40 wt % based on the total weight of the cosmetic composition. The compositions preferably include the at least one additional elastomer in an amount ranging from about 0.1 wt % to about 10 wt % based on the total weight of the cosmetic composition, such as, for example, from about 0.5 wt % to about 6 wt %, or from about 1 wt % to about 6 wt %. In some embodiments, the compositions include the at least one additional elastomer in an amount of about 1 wt % based on the total weight of the cosmetic composition.

The at least one additional elastomer may be in the form of a powder, a solution, a suspension, a dispersion, a paste, a gel, or any other suitable form. The at least one additional elastomer may be provided within a cosmetically acceptable carrier. The at least one additional elastomer may be provided within the cosmetically acceptable carrier at an amount within a range of from about 1 wt % to about 99 wt % based on a total weight of the at least one additional elastomer and the cosmetically acceptable carrier, such as, for example, within a range of from about 1 wt % to about 20 wt %, from about 1 wt % to about 15 wt %, from about 1 wt % to about 20 wt %, from about 5 wt % to about 20 wt %, from about 10 wt % to about 30 wt %, from about 10 wt % to about 40 wt %, or from about 10 wt % to about 50 wt % based on the total weight of the at least one additional elastomer and the cosmetically acceptable carrier. The compositions disclosed herein may include the at least one additional elastomer within the cosmetically acceptable carrier in a combined amount within a range of from about 10 wt % to about 50 wt % based on a total weight of the composition, such as, for example, from about 10 wt % to about 20 wt %, from about 10 wt % to about 30 wt %, from about 10 wt % to about 40 wt %, from about 15 wt % to about 25 wt %, from about 15 wt % to about 35 wt %, from about 20 wt % to about 30 wt %, from about 20 wt % to about 40 wt %, or from about 30 wt % to about 50 wt % based on a total weight of the composition. The compositions preferably include the at least one additional elastomer within the cosmetically acceptable carrier in a combined amount within a range of from about 10 wt % to about 20 wt %.

Such elastomers are commercially available from numerous sources including, but not limited to, Shin-Etsu Chemical Co., Ltd. (Tokyo, JP), Nagase America LLC (New York, NY), Grant Industries, Inc. (Elmwood Park, NJ), Sunjin Beauty Science (Seoul, KR), and Maian (Jandira, SP). The at least one additional elastomer may include, but is not limited to, USG-110 (dimethicone (and) dimethicone/vinyl dimethicone crosspolymer) from Shin-Etsu Chemical Co., Ltd., USG-110 (dimethicone (and) dimethicone/vinyl dimethicone crosspolymer) from Nagase America LLC, Gransil DMG-6/LC (dimethicone (and) polysilicone-11), Gransil GVL-912 (C9-12 alkane (and) polysilicone-11), Gransil DMG-2/LV-LC (dimethicone (and) polysilicone-11), Gransil EP-LS (polysilicone-11 (and) laureth-12), Gransil PM-GEL (phenyl trimethicone (and) polysilicone-11), or Gransense TC-8X (coco-caprylate/caprate (and) triheptanoin (and) C9-12 alkane (and) polyurethane-100) from Grant Industries, Inc., SFGEL-M (caprylic/capric triglyceride (and) dicaprylyl carbonate (and) silica (and) dextrin palmitate (and) diisostearyl malate (and) lactobacillus ferment) or SFGEL-S (caprylic/capric triglyceride (and) dicaprylyl carbonate (and) dextrin palmitate (and) diisostearyl malate (and) lactobacillus ferment) from Sunjin Beauty Science, Silike 9040 US (isoamyl laurate (and) decyl oleate (and) silica) from Maian, or any combination thereof.

The PEG-containing dimethicone crosspolymer exhibits a synergistic relationship with the at least one organic UV filter resulting in an improved photoprotective efficacy of (e.g., a photoprotective efficacy boost for) the cosmetic composition as compared to a cosmetic composition lacking the PEG-containing dimethicone crosspolymer. Accordingly, the compositions disclosed herein may be formulated to exhibit an SPF value within a range of from about 1 to about 100, such as, for example, within a range of from about 1 to about 50, from about 5 to about 40, from about 5 to about 30, from about 10 to about 80, from about 15 to about 50, from about 15 to about 80, from about 20 to about 60, from about 30 to about 50, or from about 30 to about 90. In some embodiments, the compositions may be formulated to exhibit an SPF value within a range of from about 15 to about 50. The compositions disclosed herein are preferably formulated to exhibit an SPF value of at least about 15, such as, for example, at least about 20, at last about 25, at least about 30, at least about 40, at least about 45, or at least about 50. In some embodiments, the compositions may be formulated to exhibit an SPF value of about 15, about 20, about 25, about 30, about 35, about 40, about 45, or about 50.

The cosmetic compositions disclosed herein may further include a cosmetically acceptable base (e.g., vehicle, carrier, excipient). The cosmetically acceptable base may be in the form of a liquid, a solid, a gel, an emulsion, a cream, a lotion, a paste, an ointment, a solution, an aerosol, an anhydrous base, a powder, a milk, or any other suitable form. Any and all cosmetically acceptable bases typical in the art of cosmetic formulation are considered to be suitable. By way of non-limiting example, the cosmetically acceptable base may include water, glycerin, organic solvents (e.g., hydrocarbon solvents, volatile organic solvents, non-volatile organic solvents), mono alcohols (e.g., C1-4 alcohols), polyols, fatty alcohols, esters (e.g., fatty esters), ethers (e.g., fatty ethers), glycols, glycol ethers, alkanes (e.g., paraffins), silicone oils, vegetable oils, mineral oils, liposomes, or any combinations thereof. The cosmetically acceptable base may be formed of and/or include at least a portion of the cosmetically acceptable carrier of the at least one PEG-containing dimethicone crosspolymer and/or the cosmetically acceptable carrier at least one additional elastomer. Preferably, the cosmetically acceptable base is substantially free of water (e.g., anhydrous).

Suitable examples of mono alcohols and polyols include, but are not limited to, ethyl alcohol, isopropyl alcohol, propyl alcohol, benzyl alcohol, and phenethyl alcohol. Suitable examples of alkanes include, but are not limited to, branched alkanes, such as isododecane and isohexadecane, and linear alkanes (e.g., volatile alkanes), such as C9-21 alkanes (e.g., C9-12 alkanes, C9-15 alkanes, C12-15 alkanes, C13-15 alkanes, C15-19 alkanes). Suitable examples of silicone oils include, but are not limited to, dimethicone, dimethiconol, cyclopentasiloxane (e.g., cyclomethicone), cyclohexasiloxane, phenyl trimethicone, methyl trimethicone, caprylyl methicone, and any combination thereof. Additional suitable examples of components (e.g., constituents, ingredients) of the cosmetically acceptable base include, but are not limited to, octyldodecanol, isopropyl myristate, isopropylpalmitate, squalane, paraffin derivatives, glycerol, ethylene glycol, butylene glycol, hexylene glycol, pentylene glycol, propylene glycol, dipropylene glycol n-butyl ether, propane diol, and any combination thereof. In some embodiments, the cosmetically acceptable base includes at least one silicone oil. In some embodiments, the cosmetically acceptable base includes one or more linear alkanes.

The compositions disclosed herein may include the cosmetically acceptable base in an amount ranging from about 5 wt % to about 80 wt % based on a total weight of the cosmetic composition, such as, for example, from about 5 wt % to about 20 wt %, from about 5 wt % to about 30 wt %, from about 10 wt % to about 20 wt %, from about 10 wt % to about 40 wt %, from about 15 wt % to about 30 wt %, from about 15 wt % to about 45 wt %, from about 20 wt % to about 50 wt %, from about 30 wt % to about 60 wt %, from about 40 wt % to about 70 wt %, or from about 50 wt % to about 80 wt % based on the total weight of the cosmetic composition.

The cosmetic compositions disclosed herein may include one or more SPF boosters. As used herein, “SPF booster” means and includes a compound, an active, or a component that enhances SPF in the presence of one or more UV filters, without significantly increasing the concentration (e.g., amount, wt %) of UV filters. The one or more SPF boosters may improve dispersion of the at least one UV filter, increase absorption and retention of the at least one UV filter on skin, enhance water resistance of the at least one UV filter, provide additional UV scattering and reflection, exhibit synergistic effects with the at least one UV filter, improve stability of the at least one UV filter, and/or contribute to formation of a uniform film when the compositions are applied on skin.

The one or more SPF boosters may include, but are not limited to, antioxidants, film formers (e.g., film forming agents), emollients, waxes, reflective particles, oil soluble and hydrophilic polymer solutions, dispersions, or powders (e.g., polyurethanes, polyacryalates), or any combination thereof. By way of non-limiting example, the one or more SPF boosters may include, for example, calcium sodium borosilicate, microcrystalline cellulose, silica, silica silylate, disteardimonium hectorite, dextrin palmitate, tocopheryl acetate (vitamin E), green tea extract, caprylic/capric triglyceride, squalane, aloe vera, licorice extract, sea buckthorn oil, (PVP), vinylpyrrolidone (VP)/eicosene copolymer, triacontanyl polyvinylpyrrolidone polyamide-3, polyethylene, C30-38 olefin isopropyl maleate/maleic anhydride (MA) copolymer, sorbitol/sebacic acid copolymer behenate, styrene/acrylates copolymer, polyacrylate 15, polyacrylate 17, polyurethane 34, ethylhexyl methoxycrylene, diethylhexyl 2,6-napthalate, butylphthalimide/isopropylphthalimide, phenylethyl benzoate, or any combination thereof.

Such SPF boosters are commercially available from numerous sources including, but not limited to, Elementis (London, UK), Ashland Inc. (Wilmington, DE), Miyoshi Kasei, Inc. (Tokyo, JP), PolymerExpert (Pessac, FR), Cabot Co. (Boston, MA), and Kobo Products, Inc. (South Plainfield, NJ). The one or more SPF boosters may include, but are not limited to, Bentone Gel® ISD V (isododecane (and) disteardimonium hectorite, propylene carbonate) from Elementis, Antaron™ (Ganex™) V-220 F Polymer (VP/eicosene copolymer (and) VP/acrylates/lauryl methacrylate copolymer (and) triacontanyl PVP (and) VP/hexadecane copolymer) or Antaron™ (Ganex™) WP-660 Polymer (VP/eicosene copolymer (and) VP/acrylates/lauryl methacrylate copolymer (and) triacontanyl PVP, VP/hexadecane copolymer) from Ashland Inc., Rheopearl KL2 (dextrin palmitate) from Miyoshi Kasei, Inc., EstoGel® M (castor oil/IPDI copolymer (and) caprylic/capric triglyceride) from PolymerExpert, CAB-O-SIL® TS530 (silica silylate) from Cabot Co., MSS-500/3H (silica) from Kobo Products, Inc., or any combinations thereof.

The cosmetic compositions disclosed herein may include the one or more SPF boosters in a total amount within a range of from about 1 wt % to about 15 wt % based on a total weight of the cosmetic composition, such as, for example, from about 1 wt % to about 3 wt %, from about 1 wt % to about 5 wt %, from about 1 wt % to about 10 wt %, from about 5 wt % to about 10 wt %, from about 5 wt % to about 15 wt %, or from about 10 wt % to about 15 wt % based on the total weight of the cosmetic composition.

The compositions disclosed herein may include one or more emollients. The one or more emollients may improve or provide a desired sensory feel of the cosmetic compositions. For example, the one or more emollients may improve an initial feel of the cosmetic compositions, an after-feel of the cosmetic compositions, spreadability of the cosmetic compositions, and/or absorption of the cosmetic compositions when applied to skin.

Suitable emollients include, but are not limited to, silicone-based emollients (e.g., volatile silicones, dimethicones, cross-linked silicones), fatty alcohols, fatty acids, natural oils, natural butters, phytosterols, ceramides, alkanes (e.g., branched alkanes, linear alkanes), mineral oils, petrolatum, petroleum, waxes, tribehenin, squalane, isododecane, C13-16 isoparaffin, methyl trimethicone, cyclopentasiloxane, dimethicone, phenyl trimethicone, dimethicone crosspolymer, dimethiconol, dimethicone copolyol, C12-15 alkyl benzoate, stearyl alcohol, cetyl alcohol, oleyl alcohol, isocetyl alcohol, propane-1,2-diol, butane-1,3-diol, octadecan-2-ol, glyceryl monostearate, isopropyl isostearate, stearic acid, isostearic acid, diisopropyl adipate, isocetyl stearate, isopropyl stearate, butyl stearate, isopropyl laurate, hexyl laurate, decyl oleate, isobutyl palmitate, cetyl palmitate, isopropyl palmitate, palmitic acid, dimethylpolysiloxane, glyceryl monoricinoleate, di-n-butyl sebacate, isopropyl myristate, butyl myristate, myristyl myristate, isopropyl linoleate, lauryl lactate, myristyl lactate, polyethylene glycol, triethylene glycol, lanoline, acetylated lanolin, sesame oil, coconut oil, arrachis oil, castor oil, mink oil, microcrystalline wax, ceresin, or any combination thereof. Any and all cosmetically acceptable elastomers are suitable.

Such emollients are commercially available from numerous sources including, but not limited to, Momentive Performance Materials, Inc. (Niskayuna, NY), Presperse Co. (Somerset, NJ), Nagase America LLC (New York, NY), Innospec Performance Chemicals (Salisbury, NC), and Shin-Etsu Chemical Co., Ltd. (Tokyo, Japan). The one or more emollients may include, but are not limited to, SilSoft 034 (caprylyl methicone) from Momentive Performance Materials, Inc., Permethyl® 99A (isododecane) from Presperse Co., TMF-1.5 (methyl trimethicone) from Nagase America LLC, Finsolv® TN (C12-15 alkyl benzoate) or Finsolv® TPP (C12-15 alkyl benzoate, dipropylene glycol dibenzoate) from Innospec Performance Chemicals, DM-Fluid (KF-96) (e.g., DM-Fluid-0.65, DM-Fluid-2) (dimethicone) from Shin-Etsu Chemical Co., Ltd., or any combinations thereof.

The cosmetic compositions disclosed herein may include the one or more emollients in an amount of from about 1 wt % to about 50 wt % based on a total weight of the cosmetic composition, such as, for example, in an amount of from about 1 wt % to about 5 wt %, from about 1 wt % to about 10 wt %, from about 1 wt % to about 20 wt %, from about 5 wt % to about 10 wt %, from about 10 wt % to about 20 wt %, from about 10 wt % to about 35 wt %, from about 10 wt % to about 40 wt %, from about 10 wt % to about 50 wt %, from about 20 wt % to about 40 wt %, or from about 20 wt % to about 50 wt % based on the total weight of the cosmetic composition.

The cosmetic compositions containing the PEG-containing dimethicone crosspolymer and the at least one UV filter disclosed herein may be incorporated into and/or form any suitable cosmetic formulation and/or product. By way of non-limiting example, the cosmetic compositions may be incorporated into and/or form a sunscreen, a skin care formulation (e.g., a cream, a lotion, a toner, a cleanser, a serum, etc.), a makeup formulation (e.g., a primer, a base, a powder, a cake, etc.), a colored cosmetic formulation (e.g., foundation makeup, a blush, an eyeshadow, a concealer, a bronzer, a lip gloss, a lipstick, a tinted moisturizer, etc.), a self-tanner, or any suitable topical skin care and/or cosmetic product and/or formulation.

The cosmetic compositions disclosed herein may include one or more additional cosmetically acceptable ingredients, such as, for example, sunscreen solubilizers and/or stabilizers, moisturizing agents, astringent agents, chelating agents, additional active agents, self-tanning agents, film-formers (e.g., film producing polymers), fillers, thickening agents, gelling agents, odor absorbers, surfactants, additional emollients, preservatives, stabilizers, emulsifiers, antioxidants, humectants, fragrances, essential oils, essential fatty acids, structuring agents, silicas, silicates, vitamins, colorants (e.g., pigments, dyes), conditioning agents, waxes, powders (e.g., organic powders, inorganic powders, soft focus powders, plate-like non-spherical powders, metal powders), or any combinations thereof. Any and all cosmetically acceptable ingredients are considered to be suitable. The above-described additional cosmetically acceptable ingredients are optional components of the cosmetic compositions disclosed herein and may be omitted without materially affecting the cosmetic compositions.

The compositions disclosed herein may include one or more additional active agents. The one or more additional active agents may be any cosmetically acceptable active agents. Suitable additional active agents include, but are not limited to, moisturizing actives, agents used to treat age spots, keratoses, and wrinkles, anti-acne agents, antibacterial agents, anti-yeast agents, antifungal agents, antidandruff agents, anti-dermatitis agents, antipruritic agents, anti-irritant agents, anti-dry skin agents, anti-seborrheic agents, anti-aging agents, anti-wrinkle agents, additional sunscreen agents, antihistamine agents, skin lightening agents, depigmenting agents, would-healing agents, vitamins, and self-tanning agents.

The compositions disclosed herein may include one or more sunscreen solubilizers and/or stabilizers. Suitable sunscreen solubilizers and/or stabilizers include, but are not limited to, C12-15 alkyl benzoate, butyloctyl salicylate, ethylhexyl methoxycrylene, PPG-15 stearyl ether benzoate, and dicaprylyl carbonate. Any and all cosmetically acceptable sunscreen solubilizers and/or stabilizers are considered to be suitable.

The compositions disclosed herein may include one or more waxes. The one or more waxes may be naturally occurring or non-naturally occurring. Preferably, the one or more waxes are naturally occurring. Suitable waxes include, but are not limited to, aluminum distearate, aluminum trioctanoate, aluminum/magnesium hydroxide stearate, dibenzylidine sorbitol, dibutyl ethylhexanoyi glutamide, dibutyl lauroyl glutamide, long chain alkyl polyacrylate, stearoyl inulin, dextrin palmitate, tricontanyl/PVP, octadecene/MA copolymer, bis-PEG-12 dimethicone candelillate, sorbitol/sebacic acid copolymer behenate, poly C10-30 alkyl acrylate, polyvinyl stearate, polyethylene, polyethylene copolymers, polyamides, fatty alcohols, fatty acids, glycerides (e.g., mono-, di-, and triglycerides, for example, mono/di/tri glyceryl behenate; preferably triglycerides such as tribehenin, tristearin, trihydroxystearin, tripalmitin, and/or C18-36 acid triglyceride), natural waxes (e.g., beeswax, carnauba, candellila), hydrocarbons (e.g., ozokerite, microcrystalline, paraffin), and any combination thereof.

The compositions disclosed herein may include preservatives to provide a prolonged shelf life. Suitable preservatives include, but are not limited to, potassium sorbate, imidazolidinyl urea, p-hydroxy benzoate, sodium benzoate, benzoyl alcohol, citric acid, esters of p-hydroxybenzoic acid, cosmetically acceptable parabens (e.g., methylparaben, propylparaben, butylparaben, ethylparaben, Personal Care Products Council (PCPC), previously the Cosmetic, Toiletry, and Fragrance Association (CTFA), designation parabens), ethylhexylglycerin, phenoxyethanol, caprylyl glycol, and hexylene glycol. Other preservatives suitable for use in the sunscreen compositions are disclosed in the International Cosmetic Ingredient Dictionary and Handbook, twelfth edition, 2004, the entire disclosure of which is herein incorporated by reference. Any and all cosmetically acceptable preservatives are considered to be suitable.

The cosmetic compositions disclosed herein may optionally include a humectant. Suitable humectants include, but are not limited to, polyhydric alcohols including glycerol, polyalkylene glycols, alkylene polyols, and mixtures thereof, hyaluronic acid, urea, glycerin, sorbitol, sodium 2-pyrrolidone-5-carboxylate, soluble collagen, dibutylphthalate, and gelatin. Any and all cosmetically acceptable humectants are considered to be suitable.

The compositions disclosed herein may optionally include a fragrance in any suitable amount, such as, for example, in an amount sufficient to make the composition more appealing to a consumer. Preferably, the composition includes the fragrance in an amount of from about 0.01 wt % to about 10 wt % based on a total weight of the cosmetic composition.

The following examples serve to illustrate the invention, and are not intended to, nor should they be construed to, limit the scope of the invention or be exhaustive or exclusive as to the scope of this disclosure. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance, which can be adapted to the practice of this invention in its various embodiments and the equivalents thereof.

### Example 1

### Sample Formulation

Various embodiments according to the present disclosure including the ingredients listed in Tables 1-3 of Examples 3-5 were prepared by combining the ingredients in the amounts listed to form exemplary samples S1-S6 (e.g., exemplary compositions) and comparative samples C1-C4 (e.g., comparative compositions). Each of the ingredients is commercially available and may be purchased from commercial sources including, but not limited to, DSM-Firmenich Beauty & Care (Wilheminasingel, NL), Symrise AG (Holzminden, DE), the Merck Group (e.g., Merck KGaA) (Darmstadt, DE), the Azelis Co. (e.g., DeWolf Chemical) (Warwick, RI), Kobo Products Inc. (South Plainfield, NJ), Momentive Performance Materials Inc. (Niskayuna, NY), Shin-Etsu Chemical Co., Ltd. (Tokyo, JP), Nagase America LLC (New York, NY), and Dow Chemical Co. (Midland, MI).

To form exemplary samples S1-S6 and comparative samples C1-C4, octocrylene, homosalate, octisalate, and avobenzone in the amounts listed were combined with butyloctyl salicylate in the amount listed and heated to about 70° C. to form a solution. The solution was maintained at about 70° C. while mixing until the solution was visibly clear. The resulting solution was cooled to room temperature (e.g., within a range of from about 20° C. to about 25° C.).

The remaining ingredients (e.g., silica, caprylyl methicone, dimethicone/PEG-10/15 crosspolymer, PEG-10/lauryl dimethicone crosspolymer, PEG 15/lauryl dimethicone crosspolymer, dimethicone/vinyl dimethicone crosspolymer, dimethicone crosspolymer, dimethicone, squalane, methyl trimethicone, if present) in the amounts listed were combined with the respective resulting solution and mixed continuously until the composition was substantially homogeneous.

### Example 2

### Measurement and Analysis Techniques

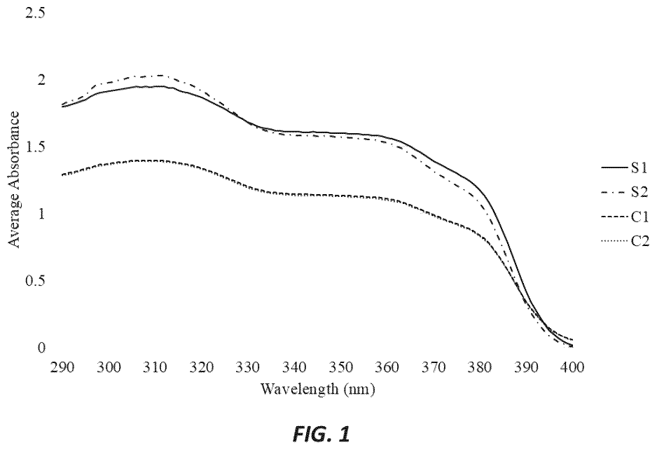

Absorption spectra for each exemplary sample and comparative sample was collected with a spectrophotometer (e.g., Labsphere UV-2000S Ultraviolet Transmittance Analyzer) by measuring a transmittance and/or absorbance of each sample as a function of wavelength of electromagnetic radiation. FIGS. 1-3 show the absorption spectra for each exemplary sample and comparative sample within a range of from about 290 nm to about 400 nm.

In vitro SPF (SPFin vitro) values, corresponding to UVB protection efficacy, and UVA protection factor (UVAPF) values were determined by calculating the SPF and UVAPF using the respective absorption spectra, according to the protocol described in ISO 24443:2021 “Cosmetics-Determination of sunscreen UVA photoprotection in vitro” published by the International Organization for Standardization in 2021 and the corrected version published in 2022, the entire disclosure of each of which is herein incorporated by reference. ISO 24443:2021 defines the term “SPFin vitro” as being the in vitro protection factor of a sun protection product against erythema-inducing radiation calculated with spectral modelling. It is readily recognized within the field of cosmetic and sunscreen formulation that SPFin vitro is indicative of relative photoprotection effectiveness of different cosmetic compositions.

To determine stability of the exemplary and comparative compositions, the compositions were held at a temperature of about 40° C. for about 24 hours. Stable compositions exhibited no substantial phase separation after about 24 hours. Unstable compositions exhibited at least some substantial phase separation after about 24 hours.

Sensory (e.g., desirable characteristics of the composition as they are perceived by the five senses, including an initial feel of the cosmetic compositions, an after-feel of the cosmetic compositions, spreadability of the cosmetic compositions, and/or absorption of the cosmetic compositions when applied to skin) of the compositions was evaluated by a sensory panel. Each composition (e.g., each exemplary and comparative composition) was assigned a sensory score between 1 and 10, with 1 exhibiting the worst sensory and 10 exhibiting the best sensory. The sensory panel determined that a sensory score equal to or greater than 2 and less than 6 was preferable and that a sensory score equal to or greater than 6 was most preferable.

### Example 3

### UV Filter(s)/PEG-Modified Elastomer Synergistic Relationship

TABLE 1
Compositions of S1, S2, C1, and C2 (wt %) and Corresponding
SPF in Vitro Values, Stability, and Sensory Scores

| Ingredient | S1 | S2 | C1 | C2 |
|---|---|---|---|---|
| Octocrylene | 9.0 | 9.0 | 9.0 | 9.0 |
| Homosalate | 7.0 | 7.0 | 7.0 | 7.0 |
| Octisalate | 4.5 | 4.5 | 4.5 | 4.5 |
| Avobenzone | 3.0 | 3.0 | 3.0 | 3.0 |
| Butyl octylsalicylate | 5.0 | 5.0 | 5.0 | 5.0 |
| Silica | 2.0 | 2.0 | 2.0 | 2.0 |
| Caprylyl methicone | 29.5 | 35.5 | 16.5 | 11.5 |
| Dimethicone/PEG-10/15 crosspolymer | 7.2 | 0 | 0 | 0 |
| PEG-10/lauryl dimethicone crosspolymer | 0 | 7.2 | 0 | 0 |
| (and) PEG-15/lauryl dimethicone | | | | |
| crosspolymer | | | | |
| Dimethicone/vinyl dimethicone crosspolymer | 1.0 | 1.0 | 8.3 | 1.0 |
| Dimethicone crosspolymer | 0 | 0 | 0 | 7.2 |
| Dimethicone | 31.8 | 9.0 | 9.0 | 49.8 |
| Squalane | 0 | 16.8 | | |
| Methyl trimethicone | 0 | 0 | 35.7 | 0 |
| SPF_{in vitro} | 87.67 | 113.33 | 24.43 | 23.56 |
| UVAPF | 26.48 | 23.28 | 10.97 | 10.7 |
| SPF_{in vitro}/UVAPF | 3.13 | 4.23 | 2.16 | 2.15 |
| Critical Wavelength (λ_{critical}) (nm) | 375.11 | 373.44 | 375.67 | 375.78 |
| Stability | Stable | Stable | Stable | Stable |
| Sensory Score | 7 | 3 | 7 | 8 |

Samples S1, S2, C1, and C2 each included the same amounts of the chemical sunscreens (e.g., octocrylene, homosalate, octisalate, avobenzone). Exemplary sample S1 included a combination of a PEG-modified dimethicone crosspolymer (e.g., dimethicone/PEG-10/15 crosspolymer) and an additional elastomer (e.g., dimethicone/vinyl dimethicone crosspolymer). Exemplary sample S2 included a combination of two PEG-modified dimethicone copolymers (e.g., PEG-10/lauryl dimethicone crosspolymer and PEG-15/lauryl dimethicone crosspolymer) and an additional elastomer (e.g., dimethicone/vinyl dimethicone crosspolymer). Comparative samples C1 and C2 were free of the PEG-modified dimethicone crosspolymer. A total amount of elastomer included in each of S1, S2, C1, and C2 was substantially the same (e.g., about 8.2). FIG. 1 illustrates absorbance spectra collected for samples S1, S2, C1, and C2 for wavelengths within a range of from about 290 nm to about 400 nm using the techniques described in Example 2. SPFin vitro values and UVAPF for each of the exemplary and comparative samples were determined using the techniques described in Example 2. As shown in Table 1 and FIG. 1, the combination of the PEG-modified dimethicone crosspolymer and the chemical sunscreens exhibited an unexpected synergistic relationship that resulted in an improved photoprotective efficacy, as evidenced by the significantly higher SPFin vitro values, UVAPF values, and UV absorbances of exemplary samples S1 and S2 as compared to the SPFin vitro values, UVAPF values, and UV absorbances of comparative samples C1 and C2.

### Example 4

### Composition Stability

TABLE 2
Compositions of S2 and C2 (wt %) and Corresponding
SPF in Vitro Values, Stability, and Sensory Scores

| Ingredient | S3 | C3 |
|---|---|---|
| Octocrylene | 9.0 | 9.0 |
| Homosalate | 7.0 | 7.0 |
| Octisalate | 4.5 | 4.5 |
| Avobenzone | 3.0 | 3.0 |
| Butyl octylsalicylate | 5.0 | 5.0 |
| Silica | 6.0 | 6.0 |
| Caprylyl methicone | 30.5 | 40.5 |
| Dimethicone/PEG-10/15 crosspolymer | 6.0 | 6.0 |
| Dimethicone/vinyl dimethicone | 1.0 | 0 |
| crosspolymer | | |
| Dimethicone | 28.0 | 19.0 |
| SPF_{in vitro} | 57.68 | 67.16 |
| UVAPF | 17.55 | 21.47 |
| SPF_{in vitro}/UVAPF | 3.20 | 2.85 |
| Critical Wavelength (λ_{critical}) (nm) | 374.56 | 375.56 |
| Stability | Stable | Unstable |
| Sensory Score | 6 | 4 |

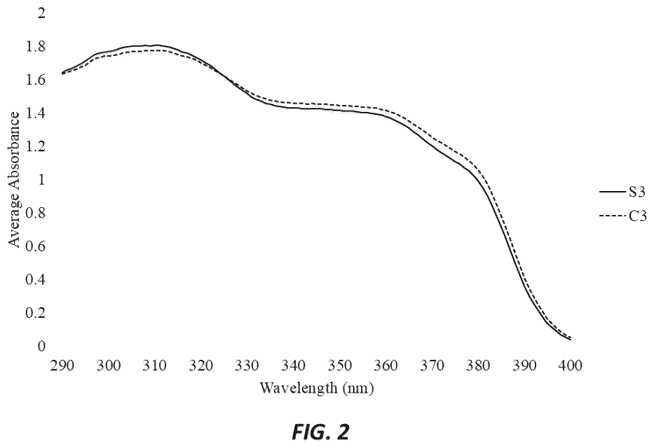

Exemplary sample S3 and comparative sample C3 each included the same amounts of chemical sunscreens (e.g., octocrylene, homosalate, octisalate, avobenzone) and PEG-modified dimethicone crosspolymer (e.g., dimethicone/PEG-10/15 crosspolymer). Exemplary sample S3 included an additional elastomer (e.g., dimethicone/vinyl dimethicone crosspolymer), while comparative sample C3 was free of an additional elastomer. FIG. 2 illustrates absorbance spectra collected for samples S3 and C3 for wavelengths within a range of from about 290 nm to about 400 nm using the techniques described in Example 2. SPFin vitro values, UVAPF values, and stability of the compositions were determined using the techniques described in Example 2. As shown in Table 2 and in FIG. 2, the chemical sunscreens and the dimethicone/PEG-10/15 crosspolymer exhibited an unexpected synergistic relationship that resulted in a slightly increased photoprotective efficacy of comparative sample C3 as compared to exemplary sample S3. It is noted that the SPFin vitro and UVAPF values of comparative sample C3 are significantly higher than those of comparative samples C1 and C2 shown in Table 1 of Example 3. Comparative sample C3 was unstable (e.g., exhibited at least some substantial separation of an oil phase), while exemplary sample S3 was stable (e.g., exhibited no substantial separation of an oil phase). The difference in stability, as defined herein, is attributed to the inclusion of the at least one additional elastomer. The SPFin vitro values of exemplary sample S3 and comparative sample C3 were determined to be within +20% of each other.

### Example 5

### Composition Sensory Analysis

TABLE 3
Compositions of S3-S5 and C4 (wt %) and Corresponding
SPF in Vitro Values, Stability, and Sensory Scores

| Ingredient | S4 | S5 | S6 | C4 |
|---|---|---|---|---|
| Octocrylene | 9.0 | 10.0 | 9.0 | 9.0 |
| Homosalate | 7.0 | 15.0 | 7.0 | 7.0 |
| Octisalate | 4.5 | 5.0 | 4.5 | 4.5 |
| Avobenzone | 3.0 | 5.0 | 3.0 | 3.0 |
| Butyl octylsalicylate | 5.0 | 5.0 | 5.0 | 5.0 |
| Silica | 2.0 | 2.0 | 2.0 | 2.0 |
| Caprylyl methicone | 19.5 | 8.0 | 4.5 | 9.5 |
| Dimethicone/PEG-10/15 | 9.6 | 9.6 | 6.0 | 14.4 |
| crosspolymer | | | | |
| Dimethicone/vinyl dimethicone | 1.0 | 1.0 | 4.0 | 0 |
| crosspolymer | | | | |
| Dimethicone | 39.4 | 39.4 | 55.0 | 45.6 |
| SPF_{in vitro} | 91.85 | 131.54 | 128.31 (film | 238.88 (film |
| | | | not uniform) | not uniform) |
| UVAPF | 32.29 | 68.95 | 36.74 | 55.64 |
| SPF_{in vitro}/UVAPF | 2.75 | 1.85 | 2.93 | 3.00 |
| Critical Wavelength | 376.11 | 378.89 | 376.67 | 377.22 |
| (λ_{critical}) (nm) | | | | |
| Stability | Stable | Stable | Stable | Stable |
| Sensory Score | 9 | 7 | 3 | 1 |

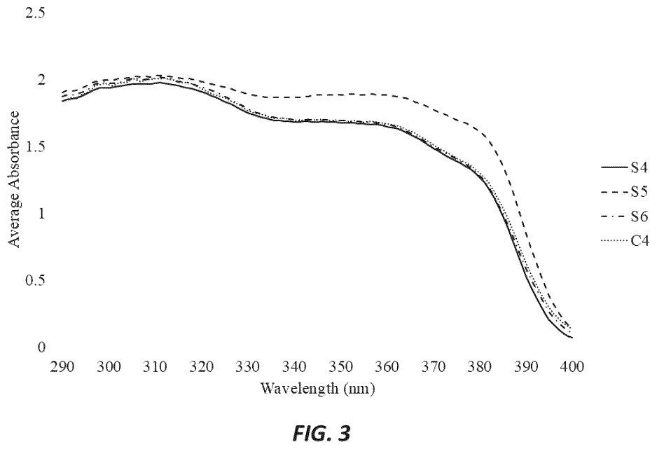

Exemplary samples S4 and S5 each included the same amounts of PEG-modified dimethicone crosspolymer (e.g., dimethicone/PEG-10/15 crosspolymer), additional elastomer (e.g., dimethicone/vinyl dimethicone crosspolymer), and dimethicone. Exemplary sample S5 included greater amounts of each of the chemical sunscreens (e.g., octocrylene, homosalate, octisalate, avobenzone) as compared to exemplary sample S4. FIG. 2 illustrates absorbance spectra collected for samples S4-S6 and C4 for wavelengths within a range of from about 290 nm to about 400 nm using the techniques described in Example 2. SPFin vitro values, UVAPF values, stability, and sensory scores for each of the compositions were determined using the techniques described in Example 2. As shown in Table 3 and in FIG. 3, the greater amounts of the chemical sunscreens of exemplary sample S5 resulted in significantly increased SPFin vitro and UVAPF values and UV absorbances as compared to exemplary sample S4. Exemplary sample S5 exhibited a slightly decreased sensory score as compared to exemplary sample S4.

Exemplary samples S4 and S6 each included the same amounts of the chemical sunscreens. Exemplary sample S6 included a lower amount of dimethicone/PEG-10/15 crosspolymer and a greater amount of dimethicone/vinyl dimethicone crosspolymer as compared to exemplary sample S4. As shown in Table 3, the decreased amount of PEG-modified dimethicone crosspolymer and the increased amount of the additional elastomer of exemplary sample S6 resulted in a similar (e.g., within +10%) SPFin vitro value to that of exemplary sample S5, which included the increased amounts of the chemical sunscreens. However, while exemplary sample S6 was functional as a sunscreen composition, exemplary sample S6 was assigned a substantially lower sensory score than both exemplary samples S4 and S5.

Exemplary sample S4 and comparative example C4 each included the same amounts of the chemical sunscreens. Exemplary sample S4 included dimethicone/vinyl dimethicone crosspolymer, while comparative example C4 was substantially free of dimethicone/vinyl dimethicone crosspolymer. Comparative example C4 included a greater amount of dimethicone/PEG-10/15 crosspolymer as compared to exemplary sample S4. As shown in Table 3, comparative example C4 was assigned a sensory level of 1, while exemplary samples S4-S6 were each assigned sensory levels greater than or equal to 3. Accordingly, compositions including a combination of the PEG-modified dimethicone crosspolymer and the additional elastomer resulted in preferable sensory levels for a consumer.

Additional non-limiting examples of embodiments of the disclosure are set forth below.

Embodiment 1: An anhydrous cosmetic composition comprises a cosmetically acceptable base, at least one organic ultraviolet (UV) filter, a polyethylene (PEG)-containing dimethicone crosspolymer, and an additional elastomer. The additional elastomer is substantially different from the PEG-containing dimethicone crosspolymer. The composition comprises the PEG-containing dimethicone crosspolymer in an amount within a range of from about 1% by weight (wt %) to about 25 wt % based on a total weight of the cosmetic composition.

Embodiment 2: The anhydrous cosmetic composition of Embodiment 1, wherein the at least one organic UV filter comprises at least four organic UV filters.

Embodiment 3: The anhydrous cosmetic composition of Embodiment 2, wherein the at least four organic UV filters consist of octocrylene, avobenzone, octisalate, and homosalate.

Embodiment 4: The anhydrous cosmetic composition of Embodiment 1, wherein the at least one organic UV filter is selected from the group consisting of octocrylene, avobenzone, octisalate, homosalate, and mixtures thereof.

Embodiment 5: The anhydrous cosmetic composition of any one of Embodiments 1 through 4, wherein the anhydrous cosmetic composition is substantially free of inorganic UV filters.

Embodiment 6: The anhydrous cosmetic composition of any one of Embodiments 1 through 5, comprising the at least one organic UV filter in a total amount within a range of from about 0.01 wt % to about 30 wt % based on the total weight of the cosmetic composition.

Embodiment 7: The anhydrous cosmetic composition of any one of Embodiments 1 through 6, wherein the additional elastomer comprises a silicone elastomer substantially free of PEG.

Embodiment 8: The anhydrous cosmetic composition of any one of Embodiments 1 through 7, wherein the additional elastomer is selected from the group consisting of polysilicone-11, dimethicone/vinyl dimethicone crosspolymer, dimethicone/PPG-20 crosspolymer, dimethicone/bis-isobutyl PPG-20 crosspolymer, dimethicone polyglycerin-3 crosspolymer, polyurethane-10, polyurethane-100, a silicone-alternative elastomer, and mixtures thereof.

Embodiment 9: The anhydrous cosmetic composition of any one of Embodiments 1 through 8, wherein the composition comprises additional elastomer in an amount within a range of from about 0.1 wt % to 50 wt % based on the total weight of the cosmetic composition.

Embodiment 10: The anhydrous cosmetic composition of any one of Embodiments 1 through 9, wherein the PEG-containing dimethicone crosspolymer is selected from the group consisting of PEG-12 dimethicone crosspolymer, PEG-10 dimethicone crosspolymer, dimethicone/PEG-10 crosspolymer, dimethicone/PEG-15 crosspolymer, dimethicone/PEG-10/15 crosspolymer, PEG-8, dimethicone/polysorbate 20 crosspolymer, PEG-10 dimethicone/vinyl dimethicone crosspolymer, PEG-10/lauryl dimethicone crosspolymer, PEG-15/lauryl dimethicone crosspolymer, PEG-15/lauryl polydimethylsiloxyethyl dimethicone crosspolymer, PEG-12 dimethicone/polypropylene glycol (PPG)-20 crosspolymer, PEG-12 dimethicone/bis-isobutyl PPG-20 crosspolymer, bis-vinyldimethicone/PEG-10 dimethicone crosspolymer, and mixtures thereof.

Embodiment 11: The anhydrous cosmetic composition of any one of Embodiments 1 through 10, wherein the PEG-containing dimethicone crosspolymer comprises dimethicone/PEG-10/15 crosspolymer.

Embodiment 12: The anhydrous cosmetic composition of any one of Embodiments 1 through 11, wherein the composition comprises the PEG-containing dimethicone crosspolymer in an amount ranging from about 2 wt % to about 16 wt % based on the total weight of the cosmetic composition.

Embodiment 13: The anhydrous cosmetic composition of any one of Embodiments 1 through 12, wherein the cosmetically acceptable base comprises linear alkanes.

Embodiment 14: A photoprotective cosmetic composition, comprising a cosmetically acceptable base, at least one organic UV filter, a PEG-containing dimethicone crosspolymer, and a dimethicone crosspolymer, wherein the dimethicone crosspolymer is substantially free of PEG. The composition comprises the PEG-containing dimethicone crosspolymer in an amount within a range of from about 2 wt % to about 16 wt % based on a total weight of the composition. The composition is anhydrous.

Embodiment 15: The photoprotective cosmetic composition of Embodiment 14, wherein the dimethicone crosspolymer is selected from the group consisting of dimethicone/vinyl dimethicone crosspolymer, dimethicone/PPG-20 crosspolymer, dimethicone/bis-isobutyl PPG-20 crosspolymer, dimethicone polyglycerin-3 crosspolymer, and mixtures thereof.

Embodiment 16: The photoprotective cosmetic composition of Embodiment 14 or Embodiment 15, wherein the PEG-containing dimethicone crosspolymer is selected from the group consisting of dimethicone/PEG-10/15 crosspolymer, PEG-12 dimethicone/PPG-20 crosspolymer, and mixtures thereof.

Embodiment 17: The photoprotective cosmetic composition of any one of Embodiments 14 through 16, wherein the composition exhibits a sun protection factor (SPF) of at least about 15.

Embodiment 18: The photoprotective cosmetic composition of any one of Embodiments 14 through 17, wherein the photoprotective cosmetic composition exhibits no substantial phase separation after being heated to and held at a temperature of about 40° C. for about 24 hours.

Embodiment 19: An anhydrous cosmetic composition, comprising a cosmetically acceptable base, at least one organic UV filter, dimethicone/PEG-10/15 crosspolymer, and dimethicone/vinyl dimethicone crosspolymer. The composition exhibits a SPF of at least about 15.

Embodiment 20: The anhydrous cosmetic composition of Embodiment 19, wherein the composition exhibits an SPF of at least about 30.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An anhydrous cosmetic composition, comprising:
a cosmetically acceptable base;
at least one organic ultraviolet (UV) filter;
a polyethylene glycol (PEG)-containing dimethicone crosspolymer; and
an additional elastomer, wherein the additional elastomer is substantially different from the PEG-containing dimethicone crosspolymer,
wherein the composition comprises the PEG-containing dimethicone crosspolymer in an amount within a range of from about 1% by weight (wt %) to about 25 wt % based on a total weight of the cosmetic composition.

2. The anhydrous cosmetic composition of claim 1, wherein the at least one organic UV filter comprises at least four organic UV filters.

3. The anhydrous cosmetic composition of claim 2, wherein the at least four organic UV filters consist of about 9 wt % of octocrylene, about 3 wt % of avobenzone, about 4.5 wt % of octisalate, and about 7 wt % of homosalate based on the total weight of the cosmetic composition.

4. The anhydrous cosmetic composition of claim 1, wherein the at least one organic UV filter is selected from the group consisting of octocrylene, avobenzone, octisalate, homosalate, and mixtures thereof.

5. The anhydrous cosmetic composition of claim 1, wherein the anhydrous cosmetic composition is substantially free of inorganic UV filters.

6. The anhydrous cosmetic composition of claim 1, comprising the at least one organic UV filter in a total amount within a range of from about 0.01 wt % to about 30 wt % based on the total weight of the cosmetic composition.

7. The anhydrous cosmetic composition of claim 1, wherein the additional elastomer comprises a silicone elastomer substantially free of PEG.

8. The anhydrous cosmetic composition of claim 1, wherein the additional elastomer is selected from the group consisting of polysilicone-11, dimethicone/vinyl dimethicone crosspolymer, dimethicone/PPG-20 crosspolymer, dimethicone/bis-isobutyl PPG-20 crosspolymer, dimethicone polyglycerin-3 crosspolymer, polyurethane-10, polyurethane-100, a silicone-alternative elastomer, and mixtures thereof.

9. The anhydrous cosmetic composition of claim 1, wherein the composition comprises the additional elastomer in an amount within a range of from about 0.1 wt % to about 50 wt % based on the total weight of the cosmetic composition.

10. The anhydrous cosmetic composition of claim 1, wherein the PEG-containing dimethicone crosspolymer is selected from the group consisting of PEG-12 dimethicone crosspolymer, PEG-10 dimethicone crosspolymer, dimethicone/PEG-10 crosspolymer, dimethicone/PEG-15 crosspolymer, dimethicone/PEG-10/15 crosspolymer, PEG-8, dimethicone/polysorbate 20 crosspolymer, PEG-10 dimethicone/vinyl dimethicone crosspolymer, PEG-10/lauryl dimethicone crosspolymer, PEG-15/lauryl dimethicone crosspolymer, PEG-15/lauryl polydimethylsiloxyethyl dimethicone crosspolymer, PEG-12 dimethicone/polypropylene glycol (PPG)-20 crosspolymer, PEG-12 dimethicone/bis-isobutyl PPG-20 crosspolymer, r, bis-vinyldimethicone/PEG-10 dimethicone crosspolymer, and mixtures thereof.

11. The anhydrous cosmetic composition of claim 1, wherein the PEG-containing dimethicone crosspolymer comprises dimethicone/PEG-10/15 crosspolymer.

12. The anhydrous cosmetic composition of claim 1, wherein the composition comprises the PEG-containing dimethicone crosspolymer in an amount ranging from about 2 wt % to about 16 wt % based on the total weight of the cosmetic composition.

13. The anhydrous cosmetic composition of claim 1, wherein the cosmetically acceptable base comprises linear alkanes.

14. A photoprotective cosmetic composition, comprising:
a cosmetically acceptable base;
at least one organic ultraviolet (UV) filter;
a polyethylene glycol (PEG)-containing dimethicone crosspolymer; and
a dimethicone crosspolymer, wherein the dimethicone crosspolymer is substantially free of PEG,
wherein the composition comprises the PEG-containing dimethicone crosspolymer in an amount within a range of from about 2% by weight (wt %) to about 16 wt % based on a total weight of the photoprotective cosmetic composition,
wherein the photoprotective cosmetic composition is anhydrous.

15. The photoprotective cosmetic composition of claim 14, wherein the dimethicone crosspolymer is selected from the group consisting of dimethicone/vinyl dimethicone crosspolymer, dimethicone/PPG-20 crosspolymer, dimethicone/bis-isobutyl PPG-20 crosspolymer, dimethicone polyglycerin-3 crosspolymer, and mixtures thereof.

16. The photoprotective cosmetic composition of claim 14, wherein the PEG-containing dimethicone crosspolymer is selected from the group consisting of dimethicone/PEG-10/15 crosspolymer, PEG-12 dimethicone/PPG-20 crosspolymer, and mixtures thereof.

17. The photoprotective cosmetic composition of claim 14, wherein the photoprotective cosmetic composition exhibits a sun protection factor (SPF) of at least about 15.

18. The photoprotective cosmetic composition of claim 14, wherein the photoprotective cosmetic composition exhibits no substantial phase separation after being heated to and held at a temperature of about 40° C. for about 24 hours.

19. An anhydrous cosmetic composition, comprising:
a cosmetically acceptable base;
at least one organic ultraviolet (UV) filter;
dimethicone/polyethylene glycol (PEG)-10/15 crosspolymer; and
dimethicone/vinyl dimethicone crosspolymer,
wherein the composition exhibits a sun protection factor (SPF) of at least about 15.

20. The anhydrous cosmetic composition of claim 19, wherein the composition exhibits an SPF of at least about 30.
